(19) United States
(12) Patent Application Publication
(10) Pub. No.: US 2025/0137959 A1
(43) Pub. Date: May 1, 2025

(54) IODINE GAS SENSOR AND METHOD

(71) Applicant: KING ABDULLAH UNIVERSITY OF SCIENCE AND TECHNOLOGY, Thuwal (SA)

(72) Inventors: Yu HAN, Thuwal (SA); Zhuo CHEN, Thuwal (SA); Xinglong DONG, Thuwal (SA); Jinrong WANG, Thuwal (SA)

(21) Appl. No.: 18/836,885

(22) PCT Filed: Jan. 31, 2023
(86) PCT No.: PCT/IB2023/050847

§ 371 Date: Aug. 8, 2024

Related U.S. Application Data
(60) Provisional application No. 63/307,686, filed on Feb. 8, 2022.

(51) Int. Cl. G01N 27/12; G01N 33/00

(52) U.S. Cl. CPC G01N 27/127; G01N 33/0027

(57) ABSTRACT

An iodine gas detection system is configured to detect iodine atoms, and the system includes a housing having an aperture, an iodine sensitive sensor located within the housing and configured to directly interact with the iodine atoms that enter through the aperture within the housing, a processor located within the housing and configured to process data collected from the iodine sensitive sensor, and a memory located within the housing and configured to store a result generated by the processor. The iodine sensitive sensor includes plural layers of reduced graphene oxide, rGO, disposed substantially parallel to each other, Ag nanoparticles distributed among the plural layers of rGO, and a surfactant polymer selected to be a surfactant and distributed among the plural layers of rGO.

### CROSS-REFERENCE TO RELATED APPLICATIONS

This application claims priority to U.S. Provisional Patent Application No. 63/307,686, filed on Feb. 8, 2022, entitled “ULTRASENSITIVE IODINE GAS SENSOR BASED ON MULTILEVEL GRAPHENE-BASED ASSEMBLY AND METHOD FOR MANUFACTURING THE SAME,” the disclosure of which is incorporated herein by reference in its entirety.

### BACKGROUND OF THE INVENTION

### Technical Field

Embodiments of the subject matter disclosed herein generally relate to a sensor and method for detecting an iodine gas, and more particularly, to a gas sensing material that is structured as a multi-level graphene-based assembly that includes silver nanoparticles and a polymer between graphene layers for providing a superior sensing performance toward the iodine atoms.

### Discussion of the Background

Nuclear power has been regarded as a reliable source of energy with the rapid growth of energy demand and also an energy source with almost a zero CO₂ foot print. However, the development of nuclear industry is always accompanied with the production of nuclear wastes or harmful environmental releases, which are feared by many. Among them, the isotopes of iodine (¹³¹I and ¹²⁹I with half life of ˜8 days and ˜15.7 million years, respectively) are major fission products which are seriously harmful to human health and have long-term impacts to environment. Therefore, the quest for developing an ultrasensitive iodine gas sensor for accurately determining the presence of these isotopes becomes important, not just for the safety of the public, but for the safety monitoring and early warning during both industrial nuclear fuel reprocessing and nuclear accidents.

Existing commercial iodine gas sensors have significant drawbacks. The common fuel-cell-type 12 sensors have relatively short lifetimes and suffer from a susceptibility to pollution. Other solid-oxide-type sensors are limited for wide applications due to the requirement of high-temperature operation (e.g., larger than 200° C.) for achieving an interaction between the gas molecules and the oxide surface. (see, for example, Small L. J. et al., Reversible MOF-Based Sensors for the Electrical Detection of Iodine Gas, ACS Appl. Mater. Interfaces 11 (2019) 27982-27988).

The scientific community has tried to come with improved iodine gas sensors. For example, a variety of zeolites and metal-organic framework (MOF) have been reported to be selective in the adsorption of the I₂ gas, including ZIF-8 (Sava, D. F. et al., Capture of Volatile Iodine, a Gaseous Fission Product, by Zeolitic Imidazolate Framework-8, J. Am. Chem. Soc. 133 (2011) 12398-12401; Hughes, J. T. et al., Thermochemical Evidence for Strong Iodine Chemisorption by ZIF-8, J. Am. Chem. Soc. 135 (2013) 16256-16259; Butova, V. V. The Effect of Cobalt Content in Zn/Co-ZIF-8 on Iodine Capping Properties, Inorg. Chim. Acta 492 (2019) 18-22), silica zeolites (Pham, T. C. T. et al., Capture of Iodine and Organic Iodides Using Silica Zeolites and the Semiconductor Behaviour of Iodine in a Silica Zeolite. Energy Environ. Sci. 9 (2016) 1050-1062), HKUST-1 (Sava, D. F. et al., Competitive I2 Sorption in Cu-BTC from Humid Gas Streams. Chem. Mater. 25 (2013) 2591-2596), micro-Cu₄I₄-MOF (Zhu, N. X. et al., Micro-Cu₄I ₄-MOF: Reversible Iodine Adsorption and Catalytic Properties for Tandem Reaction of Friedels-Crafts Alkylation of Indoles with Acetals. Chem. Commun. 52 (2016) 12702-12705), Zn₃ (DL-lac)₂(pybz)₂ (Zeng, M. H. et al., Rigid Pillars and Double Walls in a Porous Metal-Organic Framework: Single-Crystal to Single-Crystal, Controlled Uptake and Release of Iodine and Electrical Conductivity. J. Am. Chem. Soc. 132 (2010) 2561-2563), Zr₆O₄(OH)₄(sdc)₆ (Marshall, R. J. et al., Stereosselective Halogenation of Integral Unsaturated C-C Bonds in Chemically and Mechanically Robust Zr and Hf MOFs. Chem. Eur. J. 22 (2016) 4870-4877), TbCu₄I₄(ina)₃(DMF) (Hu, Y. Q. et al., Direct Observation of Confined I⁻ . . . I₂ . . . I⁻ Interactions in a Metal-Organic Framework Iodine Capture and Sensing. Chem. Eur. J. 23 (2017) 8409-8413), Co(bdc)_{1.5}(H₂bpz)0.5I₂·DMF (Li, G. P. et al., Increased Electric Conductivity upon I₂ Uptake and Gas Sorption in a Pillar-Layered Metal-Organic Framework. ChemPlusChem 82 (2017) 716-720), SBMOF-1 (Ca(sdb)) (Banerjee, D. et al., Iodine Adsorption in Metal Organic Frameworks in the Presence of Humidity. ACS Appl. Mater. Interfaces 10 (2018) 10622-10626), and silver-containing zeolites (Nenoff, T. M. et al., Silver-Mordenite for Radiological Gas Capture from Complex Streams: Dual Catalytic CH₃I Decomposition and I Confinement. Microporous Mesoporous Mater. 200 (2014) 297-303; Chapman, K. W. et al., Radioactive Iodine Capture in Silver-Loaded Zeolites Through Nanoscale Silver Iodide Formation. J. Am. Chem. Soc. 132 (2010) 8897-8899; Soelberg, N. R. et al., Radioactive Iodine and Krypton Control for Nuclear Fuel Reprocessing Facilities. Sci. Technol. Nucl. Install. 2013 (2013) 1-12).

Some of these developments succeeded in producing a material that is appropriate for iodine gas sensing, for example, the fabrication of a thin ZIF-8 film as a sensing layer. However, the response of this material in [1] was largely irreversible. Other approaches, such as the silver mordenite zeolite (Ag-MOR) which is prepared for iodine gas sensing, has a high operating temperature (70° C.), which restricts the wide applications (Small, Leo J. et al., Iodine detection in Ag-mordenite based sensors: Charge conduction pathway determinations. Microporous Mesoporous Mater. 280 (2019) 82-87). Besides these approaches, a few iodine gas sensors were fabricated based on different sensing materials, such as the iodine gas detecting paper by amine-functionalized fluorescent conjugated mesoporous polymers, which is convenient but still suffers from the imprecision in quantity analysis (Xu M, et al. Fluorescent conjugated mesoporous polymers with N, N-diethylpropylamine for the efficient capture and real-time detection of volatile iodine. J. Mater. Chem. A, 8 (2020) 1966-1974). The AgI₁₋ₓClₓ based-sensor was reported as monitoring an electrical signal with complete response and recovery capacity, but the response and recovery speed is relatively slow with 7 min and 1 h respectively (Clinsha, P. C. et al., Iodine sensing by AgI and AgI₁₋ₓClₓ. Electroanalysis 26 (2014) 2398-2402).

Thus, it remains a challenge to fabricate ultrasensitive iodine gas sensors for practical applications with high accuracy, fast response and recovery speed, low limit of detection (LoD), and reliable stability for repeated tests.

### SUMMARY OF THE INVENTION

According to an embodiment, there is an iodine gas detection system configured to detect iodine atoms, and the system includes a housing having an aperture, an iodine sensitive sensor located within the housing and configured to directly interact with the iodine atoms that enter through the aperture within the housing, and a processor located within the housing and configured to process data collected from the iodine sensitive sensor, a memory located within the housing and configured to store a result generated by the processor. The iodine sensitive sensor includes plural layers of reduced graphene oxide, rGO, disposed substantially parallel to each other, Ag nanoparticles distributed among the plural layers of rGO, and a surfactant polymer selected to be a surfactant and distributed among the plural layers of rGO.

According to another embodiment, there is a iodine gas sensor configured to detect iodine atoms, and the sensor includes plural layers of reduced graphene oxide, rGO, disposed substantially parallel to each other, Ag nanoparticles distributed among the plural layers of rGO, and a surfactant polymer selected to be a surfactant and distributed among the plural layers of rGO. A ratio of (1) a resistance of the iodine gas sensor measured in air with no iodine, and (2) a resistance of the iodine gas sensor measured in air with 150 ppm iodine gas is about 2.3.

According to yet another embodiment, there is a method for making an iodine gas sensor to detect iodine atoms, and the method includes forming a dispersion of graphene oxide, GO, mixing a surfactant polymer with the GO dispersion to form a mixture, adding a silver salt to the mixture to reduce the graphene oxide to obtain Ag nanoparticles, NPs, polymer, and reduced graphene oxide, rGO, in a resultant dispersion, dispersing the resultant dispersion in water to obtain a homogenous AgNPs-polymer-rGO dispersion, and depositing the AgNPs-polymer-rGO dispersion on top of interdigitated electrodes to form the iodine gas sensor.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a fabricated gas sensor according to an embodiment of the present disclosure;

FIG. 2 is a schematic diagram illustrating the supramolecular assembly of silver (Ag) nanoparticles (NPs) and poly (sodium-p-styrenesulfonate), PSS, polymer with reduced graphene oxide (rGO) sheets for iodine gas sensors according to an embodiment of the present disclosure;

FIG. 3 is a flow chart of a method for making a iodine gas sensor based on the material illustrated in FIG. 2 according to an embodiment of the present disclosure;

FIG. 4 is a schematic diagram illustrating a delta range dynamic gas distribution system for iodine gas sensing tests under dynamic measurement according to an embodiment of the present disclosure;

FIG. 5 is a schematic diagram illustrating the iodine gas sensing tests under static measurement according to an embodiment of the present disclosure;

FIG. 6A shows the high-resolution X-ray photoelectron spectroscopy (XPS) spectra of S 2p for the AgNPs-PSS-rGO film;

FIG. 6B shows the high-resolution XPS spectra of Ag 3d for the AgNPs-PSS-rGO film;

FIG. 7 shows the X-ray diffraction (XRD) patterns of rGO, PSS-rGO and AgNPs-PSS-rGO materials;

FIG. 8 shows the transmission electron microscopic (TEM) image of the AgNPs-PSS-rGO layer;

FIG. 9 illustrates a system that uses the AgNPs-PSS-rGO layer for detecting an iodine gas;

FIG. 10 shows the response curves of rGO, PSS-rGO and AgNPs-PSS-rGO materials for 100 part per million (ppm) of iodine gas under dynamic measurement at room temperature;

FIG. 11 shows the schematic diagram of iodine gas sensing mechanism of the AgNPs-PSS-rGO sensor;

FIG. 12 shows a successive 20-cycle response-recovery curve of AgNPs-PSS-rGO sensor in response to 150 ppm iodine gas under dynamic measurement at room temperature;

FIG. 13A shows a successive response-recovery curve of the AgNPs-PSS-rGO sensor in response to different iodine gas concentrations ranging from 150 ppm to 500 ppb under dynamic measurement at room temperature;

FIG. 13B shows a corresponding linear fit of the responses as a function of the iodine gas concentration ranging from 150 ppm to 500 ppb under dynamic measurement at room temperature;

FIG. 14A shows a typical response-recovery curve of AgNPs-PSS-rGO sensor in response to 400 ppm iodine gas under static measurement at room temperature;

FIG. 14B shows the feature of the characteristic two-stage response process of AgNPs-PSS-rGO sensor in response to 400 ppm iodine gas under static measurement at room temperature;

FIG. 14C shows the feature of the characteristic two-stage recovery process with a characteristic recovery peak of AgNPs-PSS-rGO sensor in response to 400 ppm iodine gas under static measurement at room temperature;

FIG. 15 shows a successive 5-cycle response-recovery curve of the AgNPs-PSS-rGO sensor in response to 400 ppm iodine gas under static measurement at room temperature; and

FIG. 16 shows a six-month aging test of the fabricated AgNPs-PSS-rGO sensing material in response to 400 ppm iodine gas under static measurement at room temperature.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to an iodine gas sensor based on a AgNPs-PSS-rGO assembly. However, the embodiments to be discussed next are not limited to this assembly, but may be applied to derivates of this assembly.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

It will be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

According to an embodiment, a novel ultrasensitive iodine gas sensor, which is able to detect an iodine gas under both dynamic and static atmosphere conditions, has a multilevel graphene-based assembly as the gas sensing material. The multilevel graphene-based assembly may include (1) silver nanoparticles, AgNPs, (2) a polymer, for example, poly (sodium-p-styrenesulfonate), PSS, and (3) reduced graphene oxide (rGO), which results in the AgNPs-PSS-rGO assembly. This assembly may be prepared by supramolecular assembly. In one application, the as-prepared sensor exhibits superior sensing performance toward the iodine gas with a high response (Rₐ/R_{g}=2.28, 150 ppm), reliable repeatability (˜20 successive cycles with standard deviation of 3.37%), excellent linear sensibility over the large concentration range from 500 ppb to 150 ppm, and fast response/recovery speed (˜22 s under dynamic measurement, and less than 20 s under static measurement). The AgNPs-PSS-rGO sensing material reveals the characteristic sensing peak toward iodine gas that is desired to ensure the gas selectivity. High performance, easy fabrication, and low-cost of the proposed multilevel graphene-based sensing materials/sensor is a promising element for the development of iodine gas sensors.

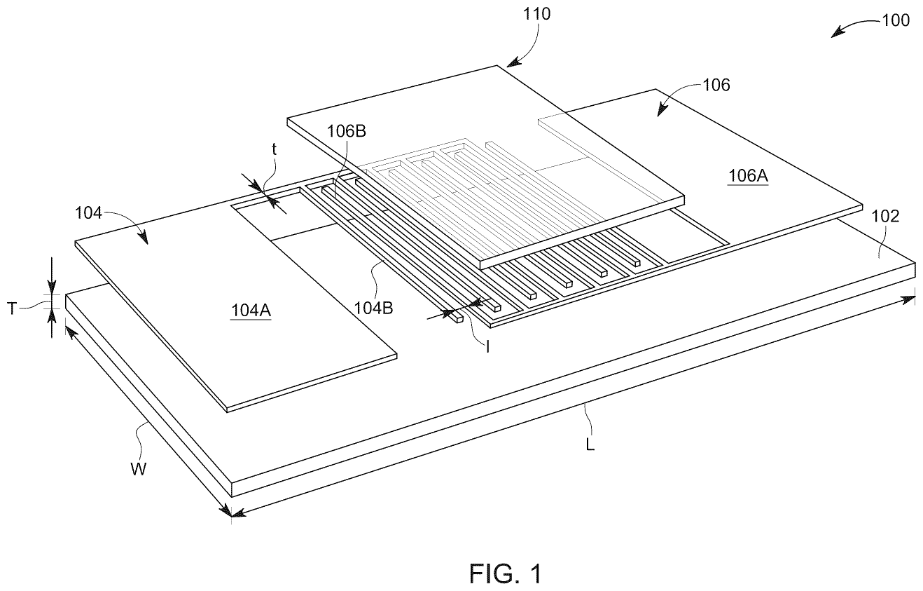

More specifically, an ultrasensitive iodine gas sensor 100 (called “the sensor” herein) is schematically illustrated in FIG. 1 and includes a substrate 102 (for example, a ceramic substrate), interdigitated electrodes 104 and 106 (e.g., made of Ag—Pd materials), and a sensing material or film 110, made of AgNPs-PSS-rGO. In one application, a length L of the substrate 102 may be about 13 mm, a width W may be about 7 mm, and a thickness T may be about 0.5 mm. Other values may be selected for the L, W and T. The interdigitated electrodes 104 and 106 include a corresponding pad 104A and 106A, and corresponding fingers 104B and 106B. A horizontal thickness t of each finger may be about 0.2 mm and a distance I between two consecutives fingers may be about 0.2 mm. In this application, the term “about” is understood to mean up to plus or minus 20% of the value that is characterized by the term.

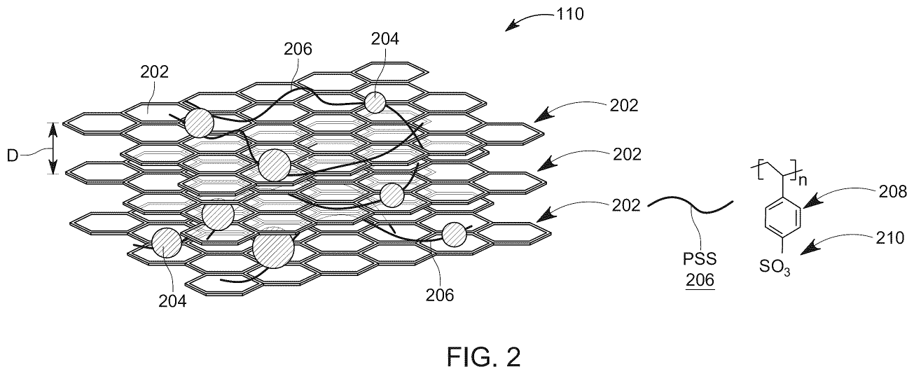

A detailed structure of the sensing material 110 is shown in FIG. 2 and includes plural rGO layers 202, which form the scaffold of the material. The plural rGO layers are arranged to be substantially parallel to each other, for at least a portion of the material 110. This means that a distance D between adjacent rGO layers is substantially constant along that portion of the material. Between the plural rGO layers, there are Ag nanoparticles 204, which are also called the “guests.” Note that these particles ensure that the rGO layers remain spaced apart from each other, which will help, as discussed later, with increasing the sensitivity detection as the iodine gas is allowed to enter between these layers. Further, a polymer 206 is present between the rGO layers 202, and this polymer acts as a bridge between the layers. The polymer 206 is selected to be a surfactant, i.e., to reduce the surface tension or the interfacial tension between the rGO layers. FIG. 2 shows the polymer 206 being the PSS polymer, which includes a benzene circle 208 and a sulfonate group 210. Other chemical structures or groups may be used for different polymers. In one application, each AgNP 204 directly bonds/engages with the polymer 206.

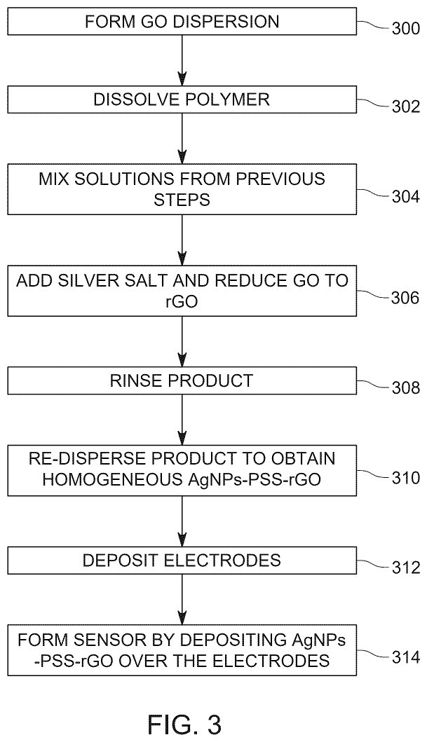

A method for making the sensor 100 is now discussed with regard to FIG. 3. The method includes a step 300 of forming a GO dispersion by ultrasonicating GO flakes in deionized (DI) water, for about 45 minutes, to prepare 0.2 mg/ml GO dispersion. Note that the numbers used in this embodiment are to provide to those skilled in the art one possible implementation of the invention, but these numbers may be varied as necessary, i.e., the amounts may be scaled up or down depending on the size of the sensor and other desired characteristics. In step 302, about 80 mg of PSS is dissolved in about 10 ml DI and in step 304, the solution from step 302 is added to 4 ml of the GO dispersion from step 300. The polymer used in the synthesis of the multilevel graphene-based assembly may include not only the PSS, but also (or instead) sodium dodecyl benzene sulfonate (SDBS), sodium dodecyl sulfate (SDS), sodium dodecyl sulfonate (SDS′), sodium lauryl sulfate (SLS), polyvinylpyrrolidone (PVP), sodium glycocholate hydrate, and/or dioctyl sulfosuccinate. However, the kind of the polymer is not limited to a specific class, as long as the polymer is a surfactant.

In step 306, 16 mg of silver nitrate, 5 mL of NaOH solution (4 mg/mL) and 10 mL of hydrazine hydrate (1 μL/mL) were added in the resultant dispersion, successively, under mild stirring, and the mixture was then stirred at 80° C. for 1 h in an oil bath. The NaOH and the hydrazine hydrate are added to reduce the GO to rGO. The silver nitrate may be replaced with silver fluoride or silver perchlorate or other silver salts that can provide the silver ions. Note that other chemical materials may be used for this step for reducing the GO. After cooling to ambient temperature, the resultant dispersion was rinsed twice in step 308 by vacuum filtration with DI water and finally re-dispersed in step 310 into 20 mL DI water under mild sonication to get a homogeneous AgNPs-PSS-rGO dispersion.

Next, in step 312, the Ag—Pd interdigital electrodes (IEs) 104, 106 (for example, 6 pairs of digits with 0.2 mm fingers and 0.2 mm gaps) are deposited on the alumina ceramic substrate 102 through a metal-jetting system. A drop and dry method is applied in step 314, where 10 μL of the AgNPs-PSS-rGO dispersion is dropped on the surface of IEs 104, 106, and then dried on a heating holder in air at 70° C. for 1 min. After this step, the thin sensing film/material 110 was formed and the gas sensor 100 is available for tests. The area of the sensing film 110 may range from 0.01 mm² to 25 cm², and a thickness of the sensing film 110 may range from 2 nm to 3 μm.

With regard to this method, it is noted that the aromatic polymer 206 was selected in this embodiment to disperse into the GO aqueous dispersion, as the polymer 206 includes benzene rings 208 which offer the intrinsic driving force to π-π interact with graphene and negatively charged groups (—SO₃⁻) 210 to enhance its hydrophilicity. Besides, the Ag⁺ was also added to the mixture and electrostatically interacted with the negatively charged groups on GO and PSS as shown in FIG. 2. After chemical reduction, the PSS polymer was assembled with the rGO nanosheets and Ag⁺ was in situ reduced to Ag nanoparticles, forming the multi-level assembly AgNPs-PSS-rGO 110 for iodine gas sensing.

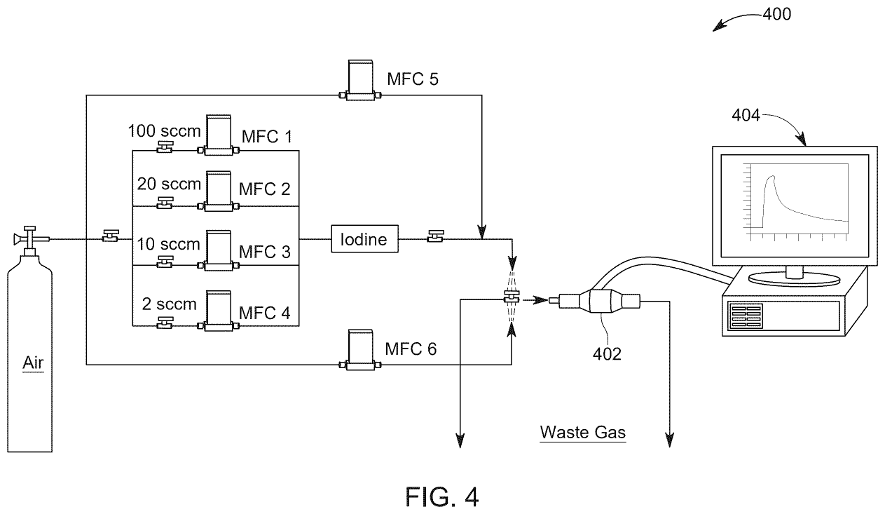

The inventors have investigated the sensor 100 under both dynamic and static conditions for gas sensing capabilities as now discussed. For the dynamic gas sensing test, a delta range dynamic distribution system 400 was used, as shown in FIG. 4., and this system includes four flowmeters (MFC 1-MFC 4) with a measuring range from 2 sccm to 100 sccm. Flowmeters MF5 and MFC 6 were used to dilute the iodine gas and replace the gas chamber 402 by air, respectively. The chamber 402 was designed to allow the quick switching between iodine gas and air. The sensor 100 is placed inside the chamber 402 and is connected to a measuring system 404, for example, a Keithley 2400 system, which is capable of simultaneously measuring a current and a voltage experienced by the sensing layer 110 of the sensor 100.

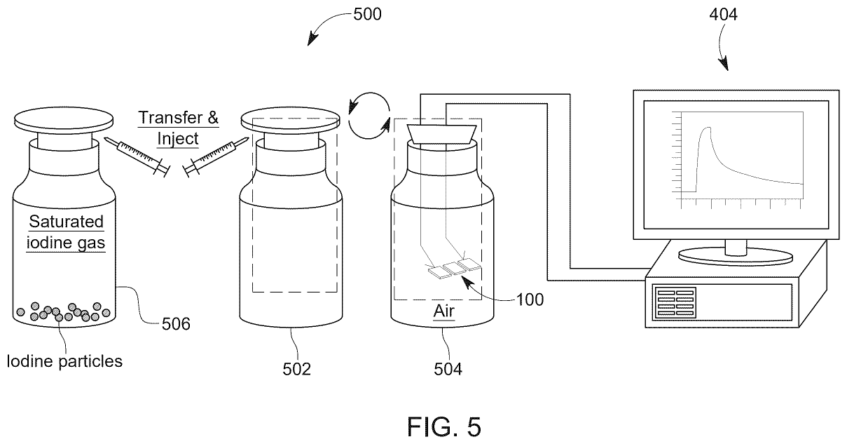

For the static conditions gas sensing test, a system 500 includes two gas collecting bottles 502 and 504, which include iodine gas and air, respectively, as shown in FIG. 5. The iodine gas, which is originally stored in a container 506 and has a testing concentration, was injected to the iodine gas collecting bottle 502, the gas sensor 100 was switched from the air bottle 504 to the iodine bottle 502 when the test started, and then switched back to the air bottle when the test ended so that there is no air flow present. These gas sensing tests were monitored by the system 404 discussed above at room temperature (˜25° C.) under 45-65% relative humidity, and each final data were obtained based on five times measurements.

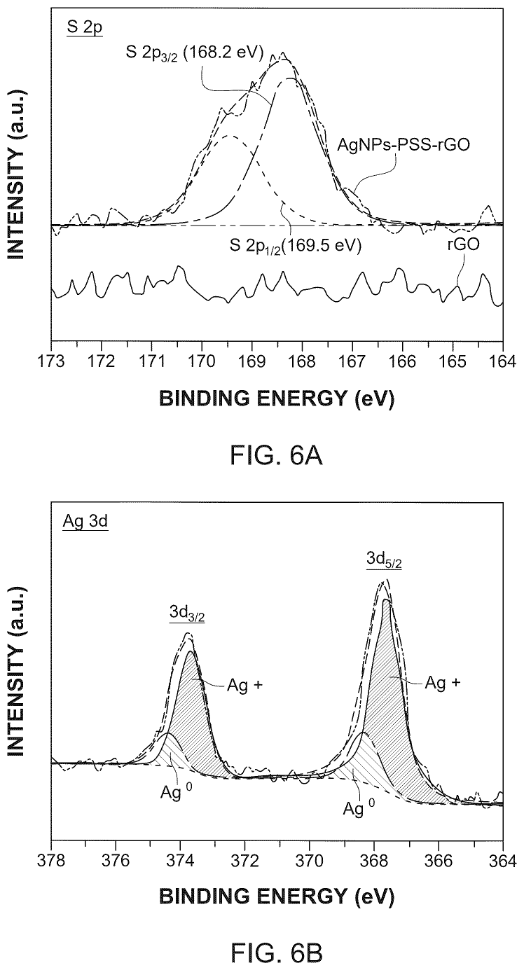

To examine the surface properties of the sensing film 110, the AgNPs-PSS-rGO material was characterized by XPS. As shown in FIG. 6A, the spectrum of the AgNPs-PSS-rGO film shows an obvious S 2p peak at 168.4 eV that is assigned to the —SO₃⁻ of PSS, indicating the successful assembly of the PSS on the graphene. As shown in FIG. 6B, the XPS spectrum also shows the characteristic Ag 3d peaks at 373.9 eV and 367.9 eV respectively, which clearly indicates that the AgNPs have been successfully assembled onto the graphene layers. In addition, the peak deconvolution revealed the contribution from both metallic (Ag⁰) and oxidized (Ag⁺) species.

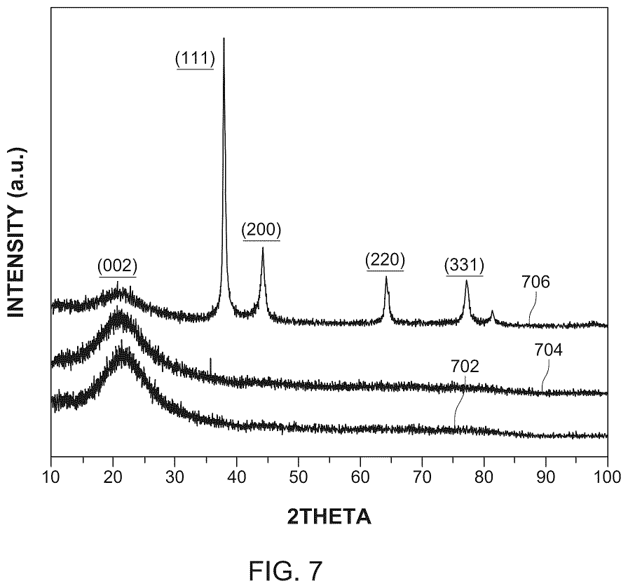

To examine the crystal quality and structure of the sensing film and its components, rGO, PSS-rGO and AgNPs-PSS-rGO materials were investigated by X-ray diffraction (XRD) as shown in FIG. 7. The XRD pattern for rGO 702 and PSS-rGO 704 exhibit a single diffraction reflection at 21.35°, which is attributed to the diffraction from the (002) plane of the hexagonal graphite structure. However, the AgNPs-PSS-rGO composite exhibits an XRD pattern 706 having several, well-defined, diffraction reflections at 20-38.07°, 44.15°, 64.34° and 77.42°, attributed to the (111), (200), (220) and (311) planes respectively, which are corresponding to the face-centered cubic (fcc) structure of the AgNPs.

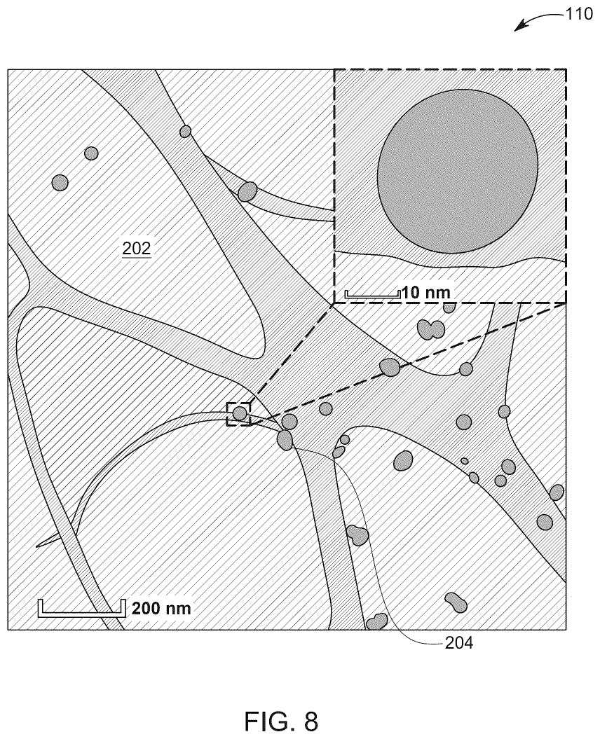

FIG. 8 shows a typical transmission electron microscopic (TEM) image of a thin layer of AgNPs-PSS-rGO material 110. AgNPs 204 with average diameters ranging from 30 nm to 60 nm were uniformly distributed on the graphene sheet 202. The same result was also examined by atomic force microscopy (AFM), not shown.

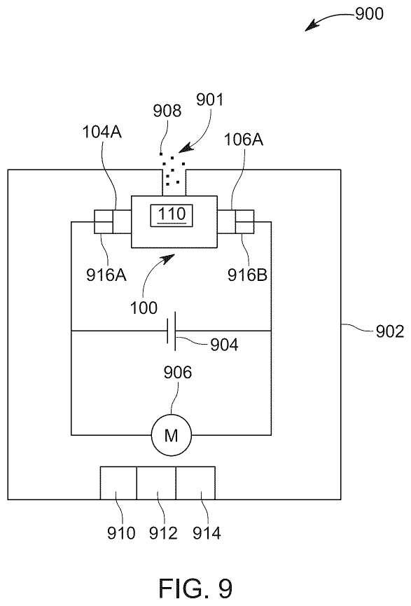

FIG. 9 shows a system 900 that includes the sensor 100 for measuring iodine gas. The system 900 has a housing 902 that holds the sensor 100, and a power source 904. The power source 904 is configured to supply power to the sensor 100, by having its terminals connected to the pads 104A and 106A, respectively. The power source may be a battery, solar cell, fuel cell, or any known power source. A measuring circuit 906 is also connected to the pads 104A and 106A for estimating the current or voltage recorded by the sensor 100 due to the interaction with the iodine gas atoms 908. The iodine atoms 908 are free to enter the housing 902, through at least one opening or aperture 901, so that they can directly interact with the sensing material 110 of the sensor 100. A processor 910, memory 912, and/or communication interface 914 may also be present in the system 900 so that the processor 910 may process the data collected by the measuring circuit 906, the memory 912 may store the various iodine gas readings detected by the sensor 100, and the communication interface 914 may, either in a wired or a wireless manner, exchange the iodine gas readings with a server (not shown) or any other device. In one application, the system 900 may be a smart device, e.g., a smart phone, and the sensor 100 may be attached to a port 916A/916B of the smart device, as the smart device already includes the power source 904, the measuring circuit 906, the processor 910, the memory 912, and the communication interface 914. Thus, the entire system 900 may be portable.

Next, the gas sensing performance of the sensor 100 was measured. In this regard, it is well known that most graphene-based materials show p-type semiconductor behavior at ambient atmosphere with holes being the main carriers, as they tend to be heavily p-doped by adsorbing O₂ and H₂O molecules in ambient air. Without exception, all the graphene-based sensing materials in this disclosure reveal p-type semiconductor behavior, and the sensing signal is shown by the variation of the resistance associated with the sensor. The response value is defined as Response=Rₐ/R_{g}, where Rₐ and R_{g} denote the resistance captured under an air and iodine gas atmosphere, respectively.

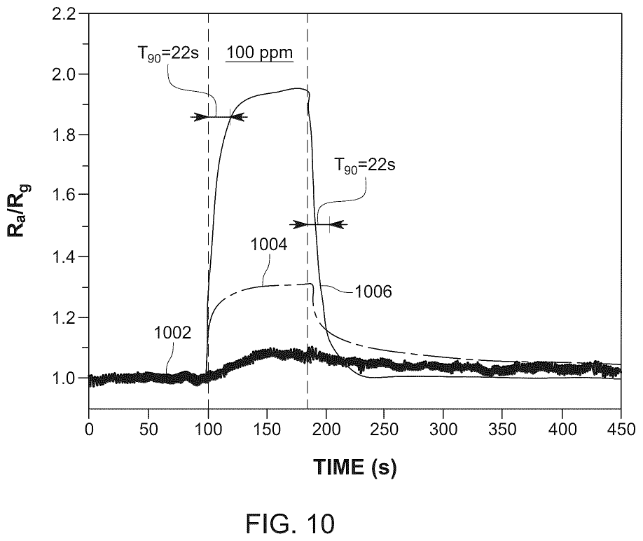

FIG. 10 shows the response curves 1002, 1004, and 1006 of rGO, PSS-rGO and AgNPs-PSS-rGO sensors, respectively, in response to 100 ppm iodine gas under dynamic measurement (i.e., using the system 400 in FIG. 4), with response value of 1.09, 1.31 and 1.93 respectively. The level-by-level sensing enhancement of AgNPs-PSS-rGO could be attributed to the synergistic effect of PSS molecules and AgNPs. Firstly, the PSS is known as a hydrophilic polymer due to the abundant sulfonate groups. After the supramolecular assembly with graphene sheets, the PSS-rGO sheets exhibit much better water dispersity than the hydrophobic rGO sheets. Thus, unlike the aggregated rGO sheets, the PSS-rGO sheets are well separated from each other, which indicates the effective sensing area of PSS-rGO sheets is larger than the rGO sheets. That is the reason why the PSS molecules contribute to the PSS-rGO to have an improved detection.

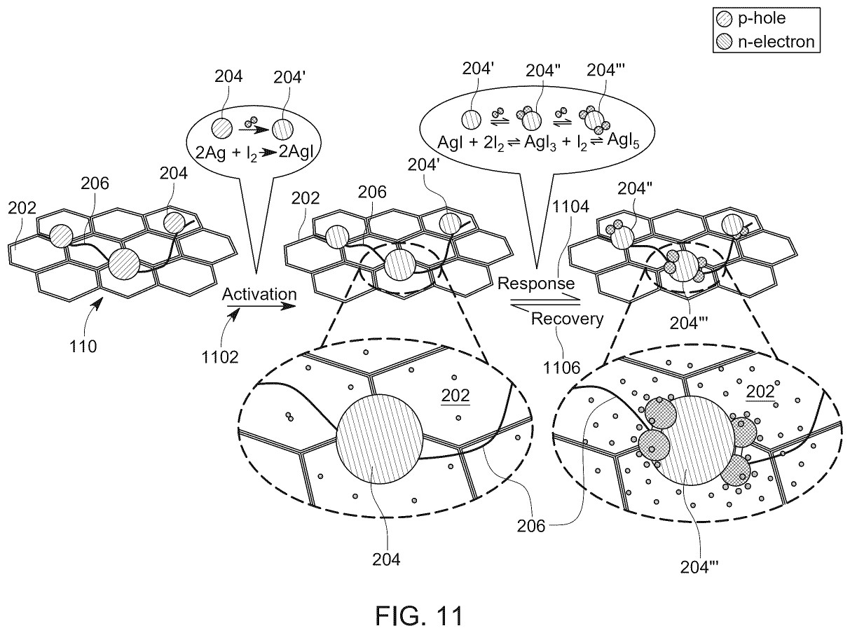

In addition, the AgNPs play a key role to enhance the iodine gas sensing performance. The Ag 204 is initially transformed to AgI 204′ after exposure to the iodine gas, and this process is defined as the activation step 1102 of the AgNPs-PSS-rGO sensor 100, as show in FIG. 11. During the following iodine gas sensing measurement step 1104, the AgI 204′ will further transform to unstable AgI₃ 204″ and AgI₅ 204″ based on the reaction AgI+2I₂ [symbol] AgI₃+I₂ [symbol] AgI₅, as shown in FIG. 11. Notably, the reaction is completely reversible so that in step 1106, the recovery step, the AgI₃ 204″ and AgI₅ 204″ go back to AgI 204′, when the sensor 100 is exposed to air. The specific and reversible reactions with iodine illustrated in FIG. 11 indicate that the sensing material can be selective for iodine gas and stable for repeated testing.

From the view of electrons transfer, the concentration of electrons (n) on the AgNPs-PSS-rGO sensor 100 decreases and the concentration of holes (p) rises during the sensing response process 1104, as shown in Equation (1) below, where nᵢ is a constant for a given semiconductor material, Eᵢ and E_{f} are the current material energy and the Fermi energy, respectively, K is the Boltzmann's constant, and T is the current temperature. Namely, the conductivity of the sensor will enhance, revealing the sensing signal in form of the decrease in resistance. On the contrary, n on the AgNPs-PSS-rGO sensor 100 will rise and p will reduce when the iodine gas leaves, revealing the sensing signal in the form of increased resistance during the recovery process 1106, completing a whole iodine sensing cycle.

$$p = \frac{{n}_{i}^{2}}{n} = {n}_{i} exp ( \frac{{E}_{i} − {E}_{F}}{KT} ) \qquad ( 1 )$$

The increased in the sensibility detection of the sensor 100 is partially due to the AgNPs 204 separating the graphene layers 202 in the multi-layer structure 110, which allows the penetration of iodine gas molecules into interlayers, accelerating the adsorption and desorption of iodine gas molecules. Thus, the multi-level structure of AgNPs-PSS-rGO further increases the effective sensing area to enhance the response value and improves the response and recovery speed.

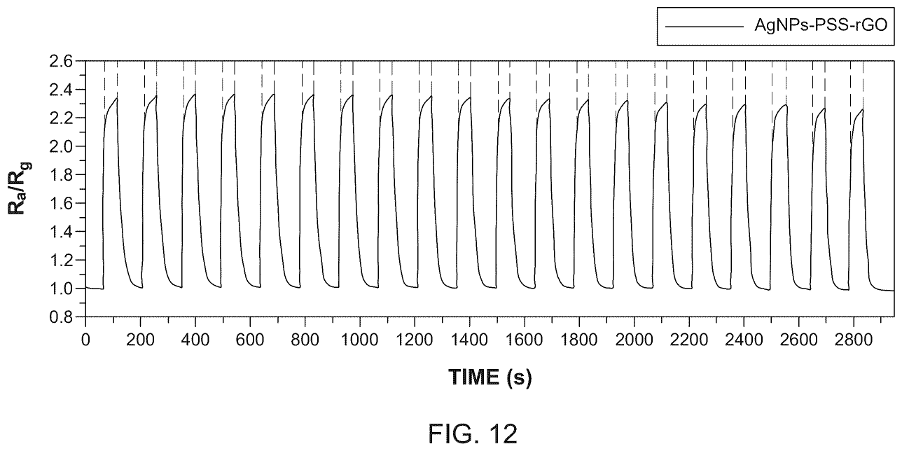

In a further test, the AgNPs-PSS-rGO based sensor 100 was exposed to 150 ppm iodine gas for 20 successive cycles under dynamic measurement. An average response (Rₐ/R_{g}=2.32) with a small standard deviation of 3.37% was measured as shown in FIG. 12, verifying the reliable repeatability of the AgNPs-PSS-rGO sensor.

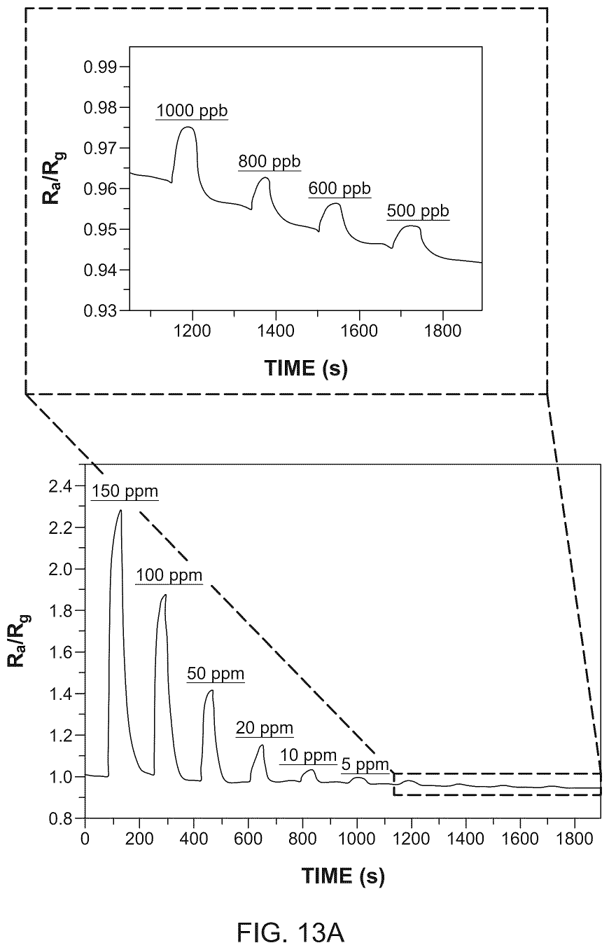

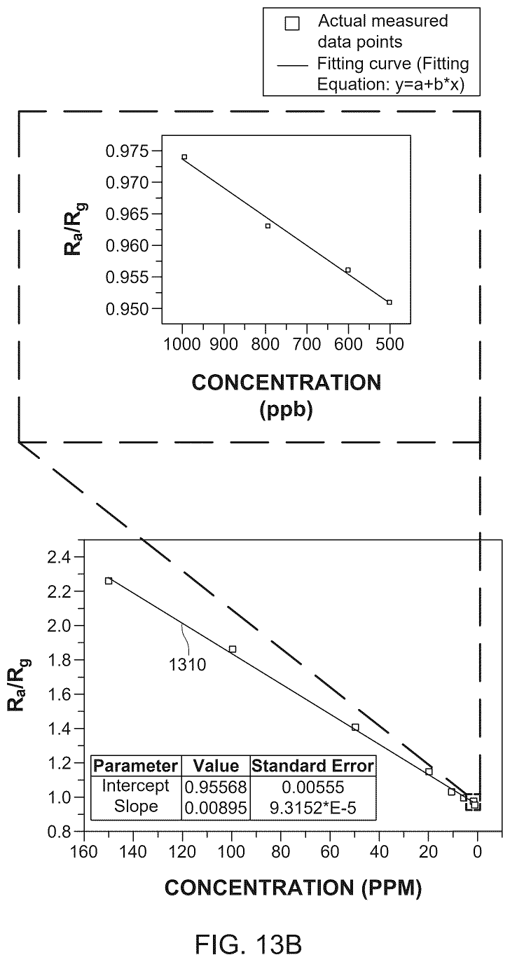

The wide-range detecting concentration and linear sensibility of an iodine sensor is desired in practical gas sensing applications. In this regard, FIG. 13A shows the successive dynamic-sensing response (Rₐ/R_{g}) of the AgNPs-PSS-rGO sensor 100 when exposed to 150 ppm to 500 ppb iodine gas. It is noted that the response amplitude decreases monotonically with the lowering concentration of the iodine gas. A linear regression is applied to find that the AgNPs-PSS-rGO sensor reveals an excellent linear detection range 1310 from 150 ppm to 500 ppb with the corresponding response (Rₐ/R_{g}) measured from 2.3 to 0.95 as shown in FIG. 13B.

For this case, the LoD of the AgNPs-PSS-rGO sensor 100 is measured to a minimum of 500 ppb. However, the actual LoD can be estimated as being 3 times of the signal noise ratio (SNR), and thus, for the small SNR of the AgNPs-PSS-rGO sensor 100, it is estimated that the LoD can be lowered to tens of ppb.

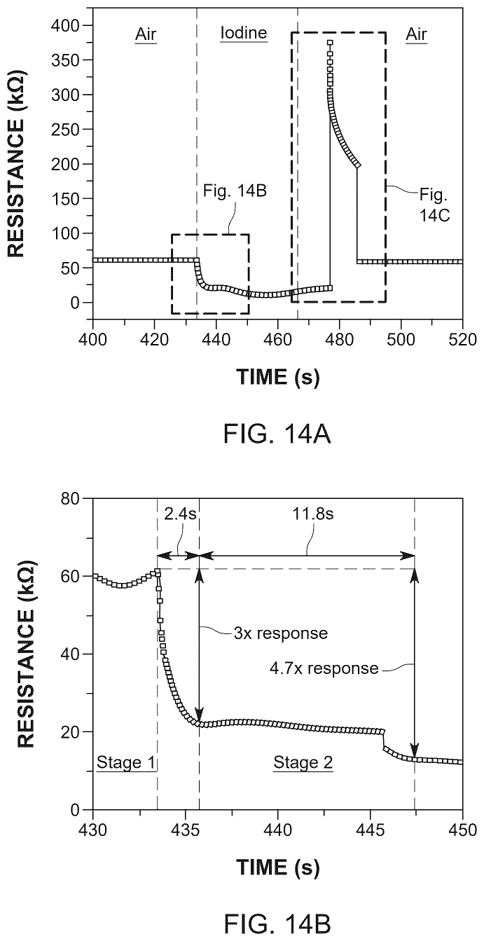

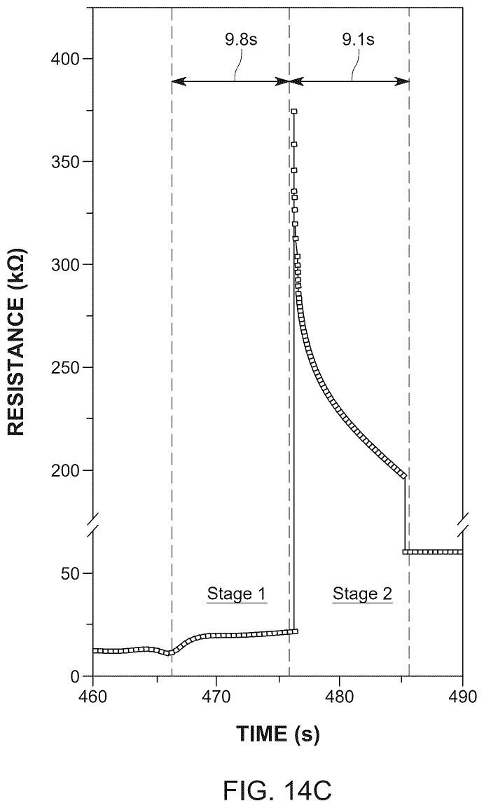

The inventors have observed that the AgNPs-PSS-rGO sensor 100 exhibits different iodine gas sensing performance under static measurement (i.e., using the system 500), as indicated by the response and recovery curve under 400 ppm iodine gas in FIG. 14A. The AgNPs-PSS-rGO sensor 100 shows a characteristic two-stage sensing process with a response value of Rₐ/R_{g}=3 and 4.7, and fast sensing speed of 2.4 s and 11.8 s respectively (see FIG. 14B). In addition, the AgNPs-PSS-rGO based sensor 100 shows a characteristic two-stage recovery process with a fast speed of 9.8 s and 9.1 s, respectively, and especially a characteristic peak in stage 2, as shown in FIG. 14C.

The reason for the different responses for dynamic measurements (FIGS. 13A and 13B) and static measurements (FIGS. 14A to 14C) could be attributed to the different adsorption and desorption mechanism of dynamic and static sensing. For dynamic measurements, the adsorption and desorption of the iodine gas are dynamic, i.e., the adsorbed iodine gas molecules will be quickly desorbed and the other gas molecules will be adsorbed as supplemented by the continuous airflow, reaching a balance under the testing concentration. However, the adsorption and desorption behavior is much different under static measurement because the testing atmosphere is static, due to being confined in a fixed space, and thus the adsorbed iodine gas molecules will not be desorbed because there is no air purge, and that corresponds to the Stage 1 of the response process. After sensing for a period of time, the absorbed iodine gas molecules will even be attached to the IEs, which corresponds to the Stage 2 of the response process. During the recovery process, the sensor is switched to the static air atmosphere, and thus a small number of the iodine molecules on the surface will get desorbed, which corresponds to the Stage 1 of the recovery process. After recovering for a period of time, the attached iodine gas molecules on the IEs eventually get desorbed. Meanwhile, the sudden desorption of the attached molecules may cause the instantaneous increase of resistance, showing a characteristic sharp peak in Stage 2. Overall, the two-stage response/recovery and the peak could be a characteristic symbol of iodine gas, which is of great significance for improving the gas selectivity.

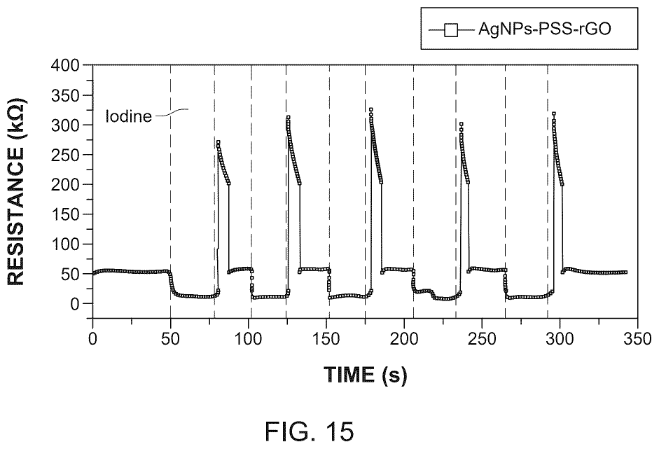

In addition, the AgNPs-PSS-rGO sensor 100 also shows reliable repeatability under static measurements, as indicated by the 5-cycle response and recovery curve shown in FIG. 15. The sensor reveals an excellent repeatability with a stable response value (Rₐ/R_{g}=4.88 with a standard deviation of 8.78%) for the tested 400 ppm iodine gas. Each cycle exhibits the two-stage response and recovery process with the characteristic peak.

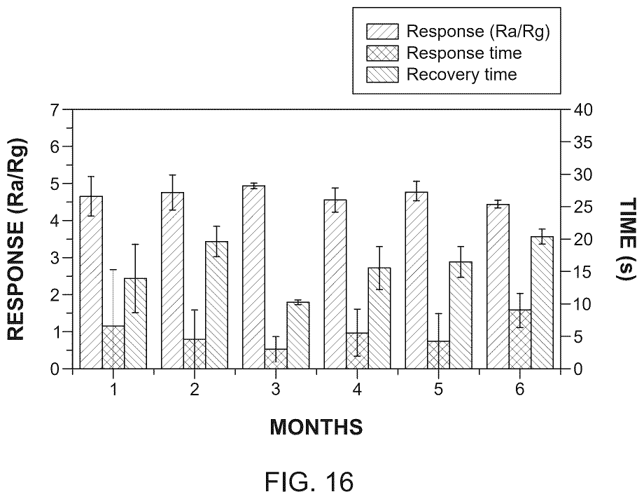

Furthermore, an aging test was carried out under static measurement conditions every month for a half year, as shown in FIG. 16, and the observed results exhibit a stable response value for 400 ppm iodine gas within 16.5% standard deviation. Moreover, the response and recovery time were also maintained around 6 s and 16 s, respectively.

Some features of the present embodiments are now summarized. The ultrasensitive iodine gas sensor is based on a multilevel graphene-based assembly i.e., AgNPs-PSS-rGO synthesized via the supramolecular assembly method. The AgNPs and PSS polymer enhance iodine sensing performance (in terms of response value, response/recovery speed, repeatability and stability) both under dynamic and static measurement.

According to an embodiment of the present disclosure, the size of AgNPs preferably ranges from 30-60 nm, but they may have a diameter of 5-200 nm. According to this embodiment or another embodiment, the AgNPs and rGO are preferably assembled with the assistance of a surfactant, for example, the PSS polymer, to prepare the multi-level graphene-based assembly. In one application, the rGO, AgNPs, and polymer in the multi-level assembly are preferably assembled at a ratio of 1:4:20 by weight during the reaction. However, the weight ratio of the rGO, AgNPs, and polymer may range from 1:0.0001:0.0001 to 1:8:40.

In one application, the hybrid mixture dispersion is preferably dropped on the Ag—Pd interdigital electrodes attached to ceramic substrate, and then dried to prepare the sensor. The area of the sensing film may range from 0.01 mm² to 25 cm², and the thickness of the sensing film may range from 2 nm to 3 μm. According to an embodiment of the present disclosure, the gas sensor with the above mixing ratio shows an unexpected high response value (Rₐ/R_{g}) of 2.28 in response to 150 ppm of iodine gas under dynamic measurement at room temperature and this is so because the specific chemical composition of the sensing material.

According to an embodiment of the present disclosure, the gas sensor preferably exhibits a LoD of 500 ppb and a linear detection range of 500 ppb to 150 ppm of iodine gas under dynamic measurement at room temperature. The gas sensor exhibits a successive iodine sensing ability of 20-cycle tests of average response (Rₐ/R_{g}=2.32, 150 ppm) with a small standard deviation of 3.37% under dynamic measurement at room temperature. In one application, the gas sensor with the above noted mixing ratio shows an unexpected response value (Rₐ/R_{g}) of 4.7 in response to 400 ppm of iodine gas under static measurement at room temperature. According to an embodiment of the present disclosure, the gas sensor exhibits a successive iodine sensing ability of 5-cycle tests of average response (Rₐ/R_{g}=4.88, 400 ppm) with a small standard deviation of 8.78% under static measurement at room temperature. According to another embodiment of the present disclosure, the gas sensor exhibits the characteristic two-stage response and recovery process with a characteristic recovery peak in response to 400 ppm iodine gas under static measurement at room temperature. In yet another application, the gas sensing materials preferably exhibit reliable sensing stability on response value with a standard deviation of 16.5% for over six months.

The disclosed embodiments provide an iodine gas sensor that is more sensitive than the existing sensors. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

The entire content of all the publications listed herein is incorporated by reference in this patent application.
[1] Small, L. J. et al., Direct Electrical Detection of Iodine Gas by a Novel Metal-Organic-Framework-Based sensor. ACS Appl. Mater. Interfaces 9 (2017) 44649-44655.

1. An iodine gas detection system configured to detect iodine atoms, the system comprising:
a housing having an aperture;
an iodine sensitive sensor located within the housing and configured to directly interact with the iodine atoms that enter through the aperture within the housing;
a processor located within the housing and configured to process data collected from the iodine sensitive sensor; and
a memory located within the housing and configured to store a result generated by the processor,
wherein the iodine sensitive sensor includes plural layers of reduced graphene oxide, rGO, disposed substantially parallel to each other, Ag nanoparticles distributed among the plural layers of rGO, and a surfactant polymer selected to be a surfactant and distributed among the plural layers of rGO.

2. The system of claim 1, wherein the surfactant polymer is poly (sodium-p-styrenesulfonate) (PSS).

3. The system of claim 1, wherein an average diameter of the Ag nanoparticles is between 30 and 60 nm.

4. The system of claim 1, wherein each Ag nanoparticle is directly bonded to the surfactant polymer.

5. The system of claim 1, wherein the surfactant polymer has a benzene ring.

6. The system of claim 5, wherein the surfactant polymer further includes a sulfonate group attached to the benzene ring.

7. The system of claim 1, wherein the iodine gas sensor further comprises:
a ceramic substrate;
interdigited electrodes directly located on the ceramic substrate; and
the iodine gas sensing layer directly located over the interdigitated electrodes.

8. The system of claim 1, wherein a ratio of (1) a resistance of the iodine gas sensor measured in air with no iodine, and (2) a resistance of the iodine gas sensor measured in air with 150 ppm iodine gas is about 2.3.

9. A iodine gas sensor configured to detect iodine atoms, the sensor comprising:
plural layers of reduced graphene oxide, rGO, disposed substantially parallel to each other;
Ag nanoparticles distributed among the plural layers of rGO; and
a surfactant polymer selected to be a surfactant and distributed among the plural layers of rGO,
wherein a ratio of (1) a resistance of the iodine gas sensor measured in air with no iodine, and (2) a resistance of the iodine gas sensor measured in air with 150 ppm iodine gas is about 2.3.

10. The sensor of claim 9, wherein the surfactant polymer is poly (sodium-p-styrenesulfonate) (PSS).

11. The sensor of claim 9, wherein an average diameter of the Ag nanoparticles is between 30 and 60 nm.

12. The sensor of claim 9, wherein each Ag nanoparticle is directly bonded to the surfactant polymer.

13. The sensor of claim 9, wherein the surfactant polymer has a benzene ring.

14. The sensor of claim 13, wherein the surfactant polymer further includes a sulfonate group attached to the benzene ring.

15. The sensor of claim 9, further comprising:
a ceramic substrate;
interdigited electrodes directly located on the ceramic substrate; and
the iodine gas sensing layer directly located over the interdigitated electrodes.

16. A method for making an iodine gas sensor to detect iodine atoms, the method comprising:
forming a dispersion of graphene oxide, GO;
mixing a surfactant polymer with the GO dispersion to form a mixture;
adding a silver salt to the mixture to reduce the graphene oxide to obtain Ag nanoparticles, NPs, polymer, and reduced graphene oxide, rGO, in a resultant dispersion;
dispersing the resultant dispersion in water to obtain a homogenous AgNPs-polymer-rGO dispersion; and
depositing the AgNPs-polymer-rGO dispersion on top of interdigitated electrodes to form the iodine gas sensor.

17. The method of claim 16, wherein a ratio of (1) a resistance of the iodine gas sensor measured in air with no iodine, and (2) a resistance of the iodine gas sensor measured in air with 150 ppm iodine gas is about 2.3.

18. The method of claim 16, wherein the polymer is poly (sodium-p-styrenesulfonate) (PSS).

19. The method of claim 18, wherein a ratio of rGO, AgNPs, and the polymer is between 1:0.0001:0.0001 to 1:8:40 by weight.

20. The method of claim 18, wherein a ratio of rGO, AgNPs, and the polymer is about 1:4:20 by weight.
